(19) United States
(12) Patent Application Publication
(10) Pub. No.: US 2026/0168995 A1
(43) Pub. Date: Jun. 18, 2026

(54) SYSTEM AND METHOD FOR A GENETIC MATERIAL TEACHING KIT

(71) Applicant: C-TAG Corporation, Boise, ID (US)

(72) Inventor: Michael Tobiason, Boise, ID (US)

(21) Appl. No.: 19/415,561

(22) Filed: Dec. 10, 2025

Related U.S. Application Data
(60) Provisional application No. 63/730,324, filed on Dec. 10, 2024.

(51) Int. Cl. G01N 33/543; G01N 21/78; G01N 33/53

(52) U.S. Cl. CPC G01N 33/54388; G01N 21/78; G01N 33/5308

(57) ABSTRACT

Genetic material teaching kits are provided which may include a manual, a genetic material sample, and a genetic material reader to demonstrate a process for identifying the source of a genetic material to a user in an engaging, game-like format.

### CROSS-REFERENCE TO RELATED APPLICATIONS

This application claims the benefit of U.S. Provisional Patent Application No. 63/730,324, entitled “SYSTEM AND METHOD FOR A GENETIC MATERIAL TEACHING KIT,” filed on Dec. 10, 2024, which is specifically incorporated by reference for all it discloses and teaches.

### TECHNICAL FIELD

The present disclosure relates generally to teaching materials and, more specifically, to a system and method for a genetic material teaching kit.

### BACKGROUND OF CERTAIN ASPECTS OF THE DISCLOSURE

Understanding key concepts about genetics and genetic material, including deoxyribonucleic acid (DNA) and ribonucleic acid (RNA), may be difficult for some students to understand conceptually. Specifically, some students may not be able to fully understand how small changes in large molecules can lead to the wide variety of life on Earth. Additionally, existing methods of teaching about genetic material tend to be overly complicated, require many hours of tedious study, and/or are too expensive for the average person to use.

Accordingly, there is a need for a genetic material teaching kit that enables a user to learn about genetic material in a fun and engaging manner.

### BRIEF SUMMARY OF SOME ASPECTS OF THE DISCLOSURE

One aspect of the present disclosure relates to a genetic material teaching kit including a manual, at least one genetic material sample, and at least one genetic material reader. The at least one genetic material reader performs an analysis on the at least one genetic material sample and a user identifies the at least one genetic material sample based on the analysis and the manual.

Another aspect of the present disclosure relates to a method of teaching a user about genetic material using a genetic material teaching kit. The genetic material teaching kit includes a manual, at least one genetic material sample, and at least one genetic material reader. The method includes conveying information about genetic material to the user using the manual, selecting the at least one genetic material sample, testing the at least one genetic material sample using the at least one genetic material reader, and determining an origin of the at least one genetic material sample based on results produced by the at least one genetic material reader.

There are other novel aspects and features of this disclosure. They will become apparent as this specification proceeds. Accordingly, this brief summary is provided to introduce a selection of concepts in a simplified form that are further described below in the detailed description. The summary and the background are not intended to identify key concepts or essential aspects of the disclosed subject matter, nor should they be used to constrict or limit the scope of the claims. For example, the scope of the claims should not be limited based on whether the recited subject matter includes any or all aspects noted in the summary and/or addresses any of the issues noted in the background.

### BRIEF DESCRIPTION OF THE DRAWINGS

A further understanding of the nature and advantages of the embodiments may be realized by reference to the following drawings. In the appended figures, similar components or features may have the same reference label.

FIG. 1 illustrates a perspective view of an example genetic material teaching kit in accordance with aspects of the present disclosure.

FIG. 2 illustrates another perspective view and partial flow diagram of the genetic material teaching kit illustrated in FIG. 1 in accordance with aspects of the present disclosure.

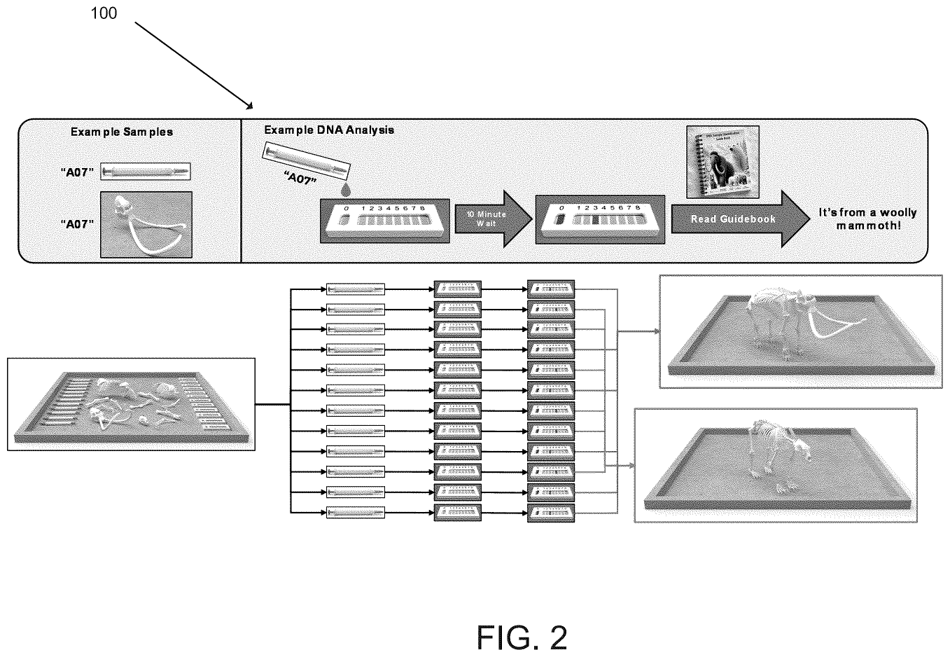

FIG. 3 illustrates another perspective view of a portion of the genetic material teaching kit illustrated in FIG. 1 in accordance with aspects of the present disclosure.

FIG. 4 illustrates another perspective view of a portion of the genetic material teaching kit illustrated in FIG. 1 in accordance with aspects of the present disclosure.

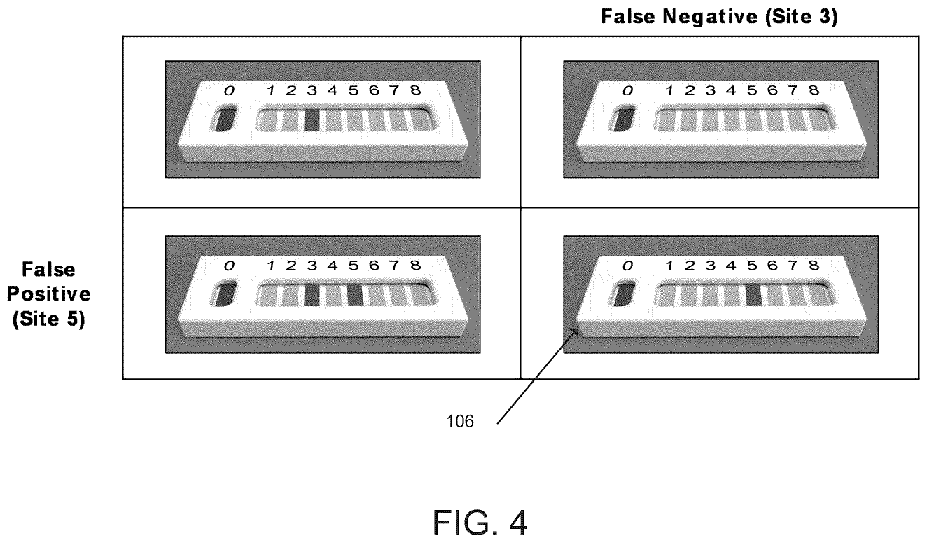

FIG. 5 illustrates examples of DNA Oligomers and Complexes for use with the example genetic material teaching kit illustrated in FIG. 1 in accordance with aspects of the present disclosure.

FIG. 6 illustrates examples of DNA Oligomers and Complexes for use with the example genetic material teaching kit illustrated in FIG. 1 in accordance with aspects of the present disclosure

FIG. 7 illustrates another perspective view of a portion of the genetic material teaching kit illustrated in FIG. 1 in accordance with aspects of the present disclosure.

FIG. 8 illustrates another perspective view of a portion of the genetic material teaching kit illustrated in FIG. 1 in accordance with aspects of the present disclosure.

FIG. 9 illustrates another perspective view of a portion of the genetic material teaching kit illustrated in FIG. 1 in accordance with aspects of the present disclosure.

FIG. 10 illustrates a perspective view of another example portion of the genetic material teaching kit illustrated in FIG. 1 in accordance with aspects of the present disclosure.

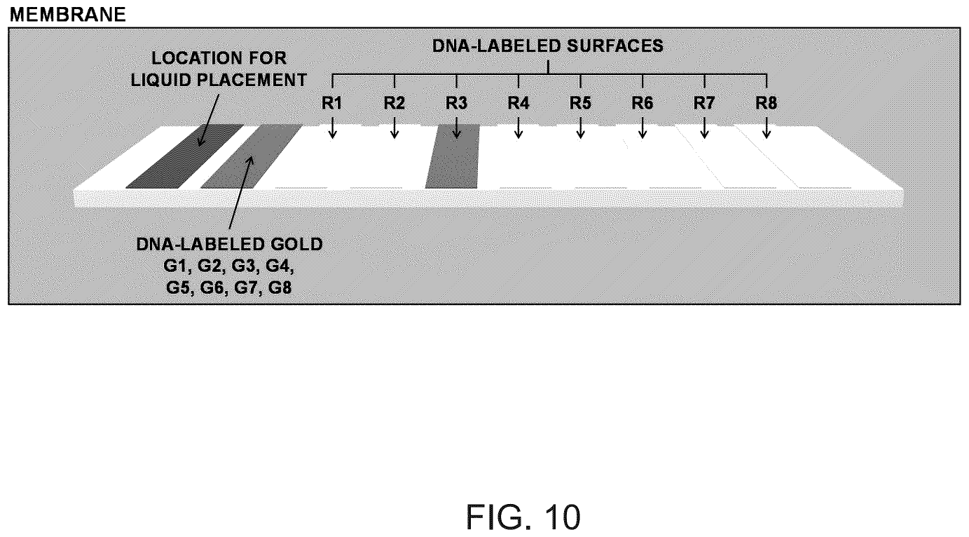

FIG. 11 illustrates examples of DNA Oligomers for use with the example genetic material teaching kit illustrated in FIG. 1 in accordance with aspects of the present disclosure.

FIG. 12 illustrates examples of DNA Complexes for use with the example genetic material teaching kit illustrated in FIG. 1 in accordance with aspects of the present disclosure.

FIG. 13 illustrates examples of DNA-Containing Liquids for use with the example genetic material teaching kit illustrated in FIG. 1 in accordance with aspects of the present disclosure.

FIG. 14 illustrates examples of DNA-Labeled Gold for use with the example genetic material teaching kit illustrated in FIG. 1 in accordance with aspects of the present disclosure.

FIG. 15 illustrates examples of DNA-Labeled Surfaces for use with the example genetic material teaching kit illustrated in FIG. 1 in accordance with aspects of the present disclosure.

FIG. 16 illustrates examples of DNA Domain Sequences for use with the example genetic material teaching kit illustrated in FIG. 1 in accordance with aspects of the present disclosure.

FIG. 17 is a flow diagram of a method of teaching a user about genetic material using the genetic material teaching kit illustrated in FIG. 1 in accordance with aspects of the present disclosure.

While the embodiments described herein are susceptible to various modifications and alternative forms, specific embodiments have been shown by way of example in the drawings and will be described in detail herein. However, the exemplary embodiments described herein are not intended to be limited to the particular forms disclosed. Rather, the instant disclosure covers all modifications, equivalents, and alternatives falling within the scope of the appended claims.

### DETAILED DESCRIPTION

The systems and methods disclosed herein relate to, among other things, a genetic material teaching kit. As described above, teaching the fundamentals of genetic material can be difficult for some students, including, in some instances, younger learners. The teaching kit described herein includes a manual, at least one genetic material sample, and at least one genetic material reader and to enable such learners to more quickly understand the basics of genetic materials in a fun and easy to understand game or exercise. The teaching kit may further include items, such as, without limitation, toys, to represent the animals to be identified during the game or teaching exercise. The manual directs the user how to use the teaching kit and explains the basics of genetic materials. The genetic material reader may be used to identify the genetic material present in each of the genetic material samples to allow the user to identify the animal corresponding to the genetic material sample. The user may then identify the animal and may match the corresponding item or toy to the genetic material sample to complete the teaching exercise or game. As such, the genetic material teaching kit described herein enables the user to understand the fundamentals of genetic material in a fun, visual, and relatively simple manner.

In some embodiments, the genetic material reader described herein may include a lateral flow assay including a plurality of detection sites. Each site includes a substance that is configured to react with a specific sequence of genetic material such that the substance can differentiate between different sets of genetic material. In the illustrated embodiment, the substance is configured to change color to indicate that a reaction has taken place. If the substance does not change color, the reaction has not taken place, and the sequence of genetic material is not found in the sample. The genetic material samples are liquid and configured to flow to each of the detection sites sequentially and the genetic material samples may each react with the substance in multiple detection sites. The manual then teaches the user how to interpret the results of the analysis conducted by the lateral flow assay and to identify the animal that each the genetic material samples corresponds to such that the user understands the fundamentals of genetic material.

Accordingly, the genetic material teaching kits described herein enable a user to learn about and understand the basics of genetic material by testing genetic material samples. More specifically, the user reads and learns about genetic material in the manual, uses the understanding gained by reading the manual to test a plurality of different genetic material samples, analyzes the results of the tests, and determines which animal the genetic material corresponds to based on the test conducted as part of the exercise or game. Reading about genetic material and conducting the exercise enables the user to understand the fundamentals of genetic material in a fun and simple manner.

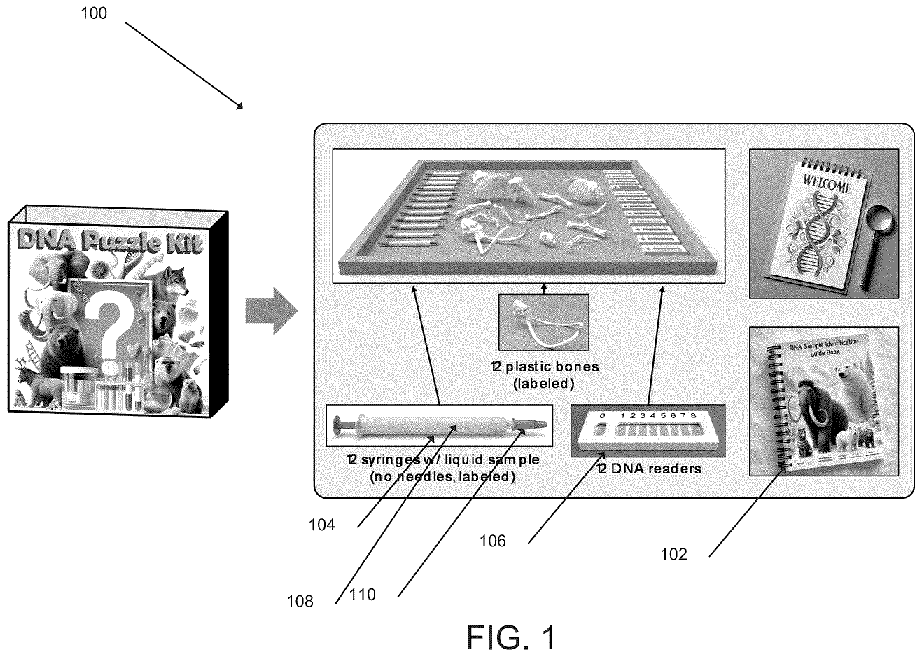

As shown in FIG. 1, the genetic material teaching kit 100 includes a manual 102, at least one genetic material sample 104, and at least one genetic material reader 106. In the illustrated embodiment, the genetic material teaching kit 100 includes a single manual 102, a plurality of genetic material samples 104, and a plurality of genetic material readers 106. Specifically, in the illustrated embodiment, the genetic material teaching kit 100 includes twelve genetic material samples 104 and twelve genetic material readers 106. In alternative embodiments, the genetic material teaching kit 100 may include any number of manuals 102, genetic material samples 104, and genetic material readers 106 that enable the genetic material teaching kit 100 to operate as described herein.

In the illustrated embodiment, the manual 102 includes a paper-based book or set of instructions that teach the user about genetic material and instruct the user how to use the genetic material teaching kit 100. In some embodiments, the manual 102 may be a book, pamphlet, spiral notebook, and/or any paper based physical booklet that includes instructions that teach the user about genetic material and instruct the user how to use the genetic material teaching kit 100. In alternative embodiments, the manual 102 may be in electronic form including, but not limited to, an electronic document, a video, electronic pictures, website, an audio file, and/or any other type of electronic medium that enables the genetic material teaching kit 100 to operate as described herein. In some cases, the manual or packaging of the teaching kit 100 could be used to provide a link, such as a QR code, to electronic resources, such as electronic versions of the manual, a corresponding website, etc.

In the illustrated embodiment, the genetic material samples 104 each include a plastic syringe 108 that each include a sample of genetic material for identification. In the illustrated embodiment, the plastic syringe 108 does not include a needle that may potentially harm young users. Rather, the plastic syringe 108 includes a plastic tip 110 that safely dispenses the sample of genetic material onto the genetic material readers 106.

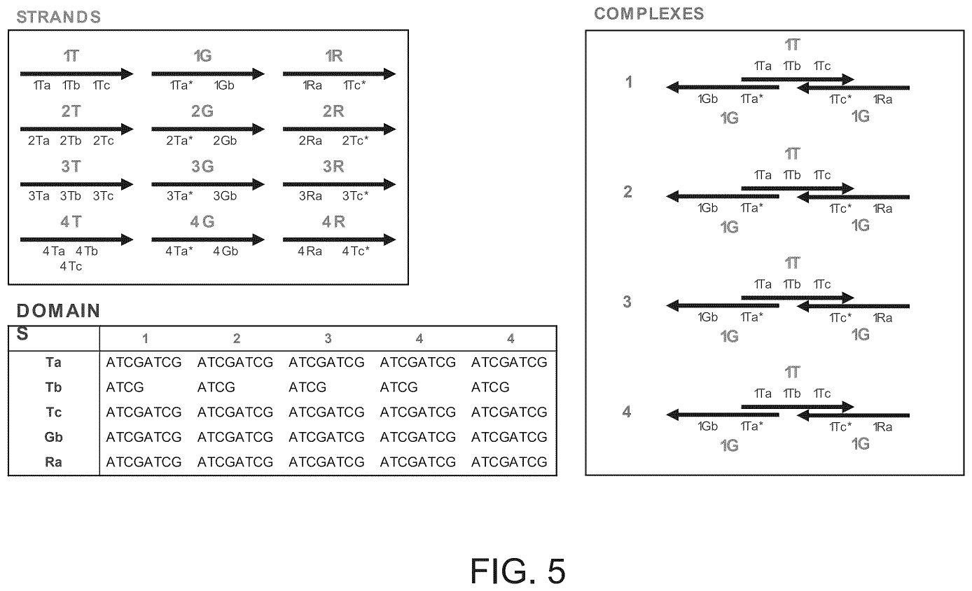

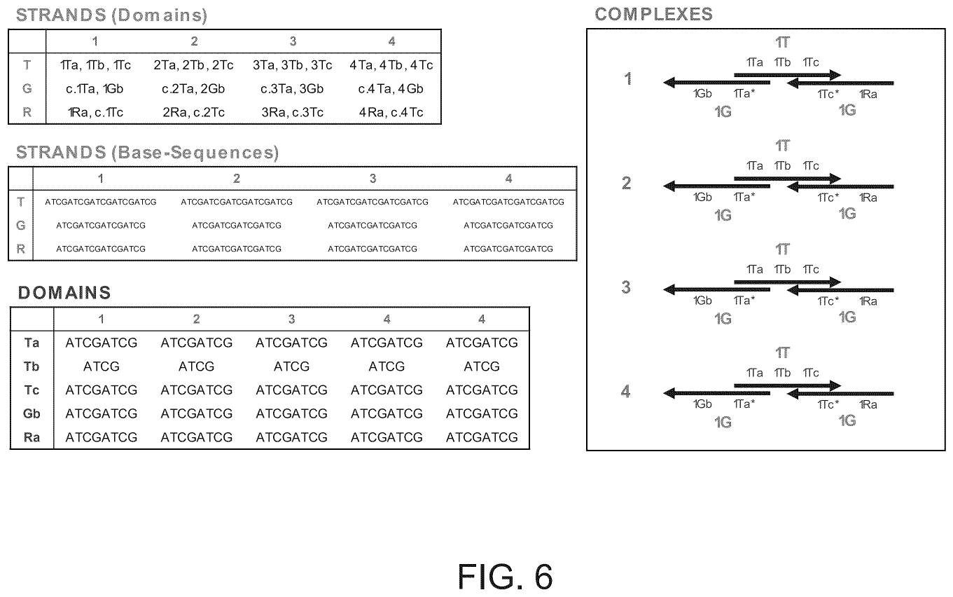

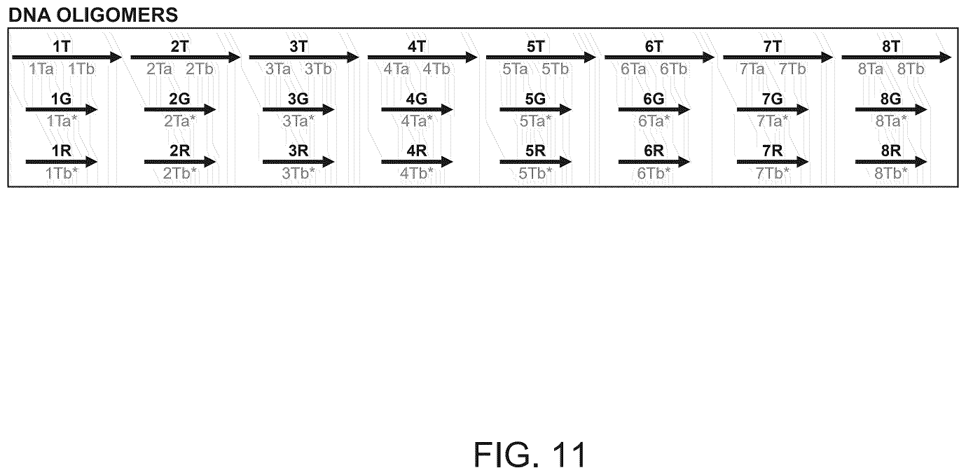

In the illustrated embodiment, the genetic material sample includes a liquid including a synthetic DNA sequence. In alternative embodiments, the genetic material sample may be any molecule that enables the genetic material teaching kit 100 to operate as described herein. In the illustrated embodiment, each genetic material sample includes one of eight different synthetic DNA samples for identification. In alternative embodiments, each genetic material sample may include any number of molecules for identification. In some embodiments, the liquid may include actual DNA sequences. For example, in some embodiments, the genetic material sample may include any of the DNA Oligomers, DNA Complexes, DNA-Containing Liquids, DNA-Labeled Gold, DNA-Labeled Surfaces, and/or DNA Domain Sequences illustrated in FIGS. 5, 6, and 11-16. In other examples, the genetic material sample may include another material which simulates DNA.

In some embodiments, the sequences provided in each genetic material sample are selected to assure that no overlapping is present to provide a clear signal as the testing is conducted.

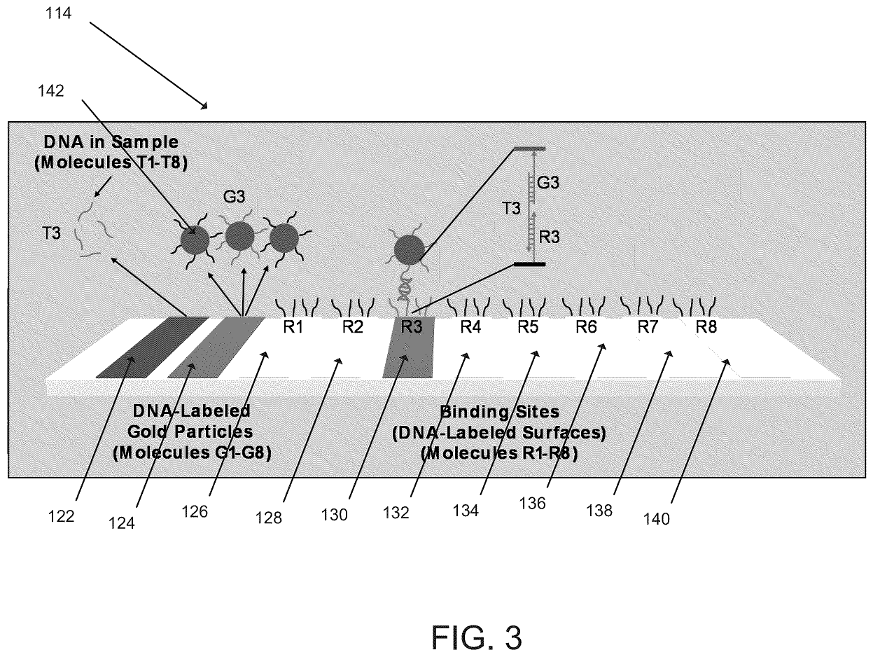

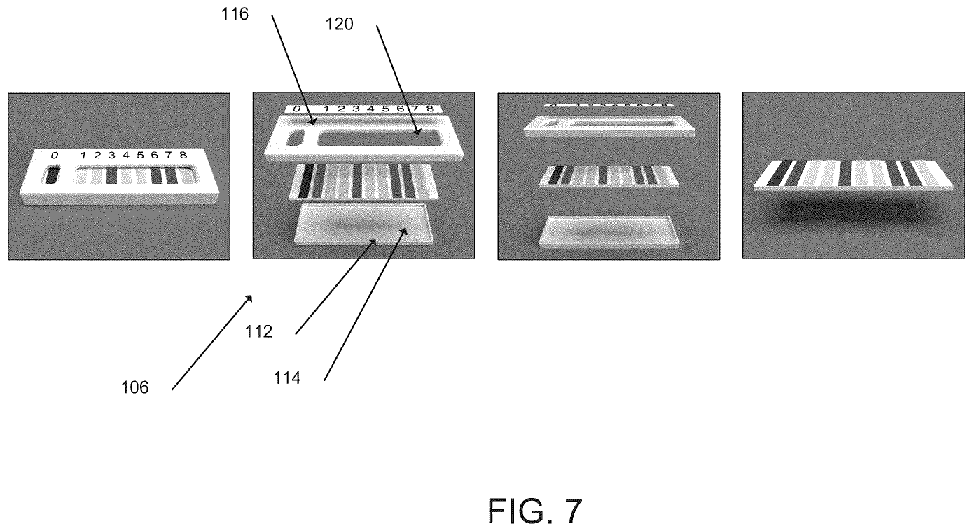

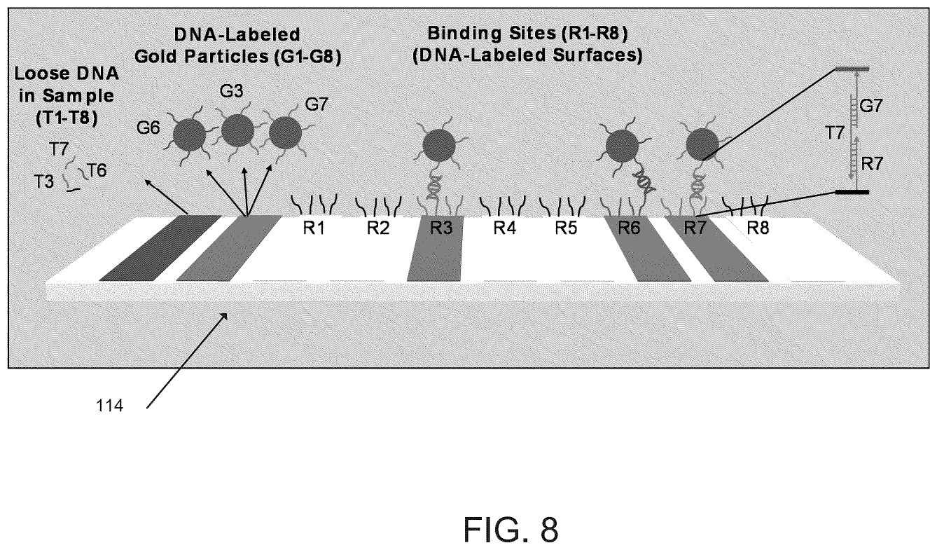

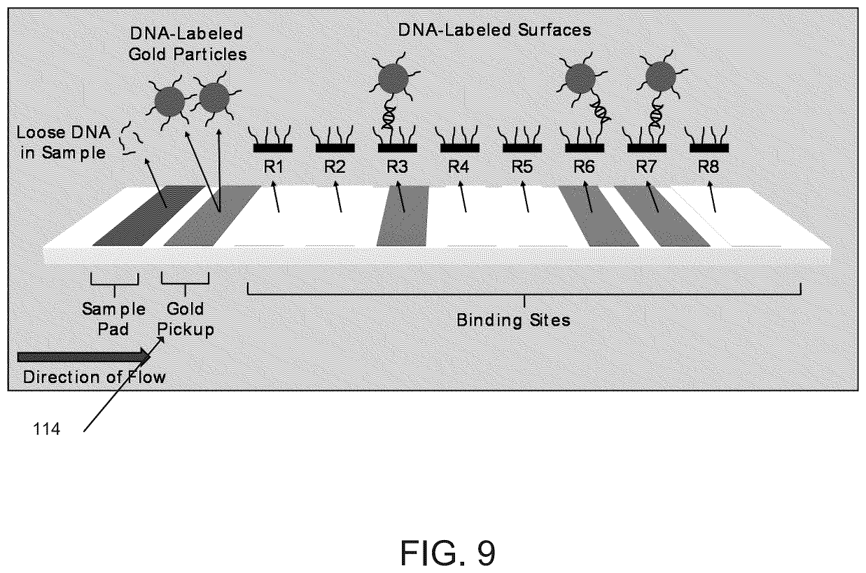

As shown in FIGS. 3 and 7-9, the genetic material readers 106 each include a multiplexed lateral flow assay configured to detect genetic material from at least one of the genetic material samples 104. In the illustrated embodiment, the genetic material readers 106 each include a cover 112 and a base 114. The cover 112 includes a lower cover 116 and an upper cover 118. The upper cover 118 defines at least one opening 120 that enables at least one of the genetic material samples 104 to be dispensed on the base 114 and enables the user to observe the base 114. The base 114 includes a sample site 122, a particle site 124, and a plurality of detection sites 126-140.

The sample site 112 is configured to receive at least one of the genetic material samples 104. The particle site 124 includes a plurality of particles 142 that are configured to be attached to the genetic material in the genetic material sample 104 when the genetic material flow through the particle site 124. In some embodiments, the plurality of particles 142 include a plurality of gold particles. In some embodiments, the plurality of particles 142 include a plurality of nanoparticles. In some embodiments, the plurality of particles 142 include a plurality of gold nanoparticles. In alternative embodiments, the plurality of particles 142 may include any type of particle that enables the sample site 112 to operate as described herein. The detection sites 126-140 each include a substance that is configured to react with a specific sequence of genetic material such that the substance can differentiate between different sets of genetic material. In the illustrated embodiment, the substance is configured to change color to indicate that a reaction has taken place. If the substance does not change color, the reaction has not taken place, and the sequence of genetic material is not found in the sample. The genetic material samples are liquid and configured to flow to each of the detection sites 126-140 sequentially and the genetic material samples may each react with the substance in multiple detection sites. In some embodiments, the substance may include synthetic DNA sequences. In some embodiments, the substance may include actual DNA sequences. For example, in some embodiments, the substance may include any of the DNA Oligomers, DNA Complexes, DNA-Containing Liquids, DNA-Labeled Gold, DNA-Labeled Surfaces, and/or DNA Domain Sequences illustrated in FIGS. 5, 6, and 11-16. In still others, the genetic material sample may include another material which simulates DNA.

In the illustrated embodiment, the base 114 includes a relatively flat rectangular strip. In alternative embodiments, the base 114 may be formed in other shapes and geometries. The base 114 may be made from any suitable material including paper or wood byproducts, plastic or polymers, organic matter such as a fabric, woven material, cellulose, cutin, tannin-based material, metal, ceramic, or vinyl. One available and inexpensive option may include paper, such as to allow for maximum liquid absorption with clear result readings. Other examples, in addition to the material named above, may include a matrix of paper or plastic, for example, or any combination of the above-mentioned materials.

Detection sites 126-140 include detection sites on a surface of the base 114 that will change, in physical attribute (change color), when brought into contact with a sequence of genetic material to which they are reactive. That is, the presence of a particular sequence of genetic material will result in the physical change in the detection sites 126-140; thereby enabling detection of the sequence of genetic material by observing the change in the detection sites 126-140. One example of a color change may be from white to pink or red. However, the disclosure is not limited to this example. Rather, the detection sites 126-140 may be constructed to be reactive to a desired substance. Additionally, such that the physical manifestation of the reaction can be in any selected form such as a change in color, odor, texture, or fluorescence. Further, the number of detection sites on the base 114 is not limited to 8, as depicted in FIGS. 3 and 7-9 with respect to detection sites 126-140. Rather, the number of detection sites on the base 114 may be less than 8 or more than 8. For example, the number of detection sites on the base 114 may include up to 64, 128, 256, 512, or more detection sites.

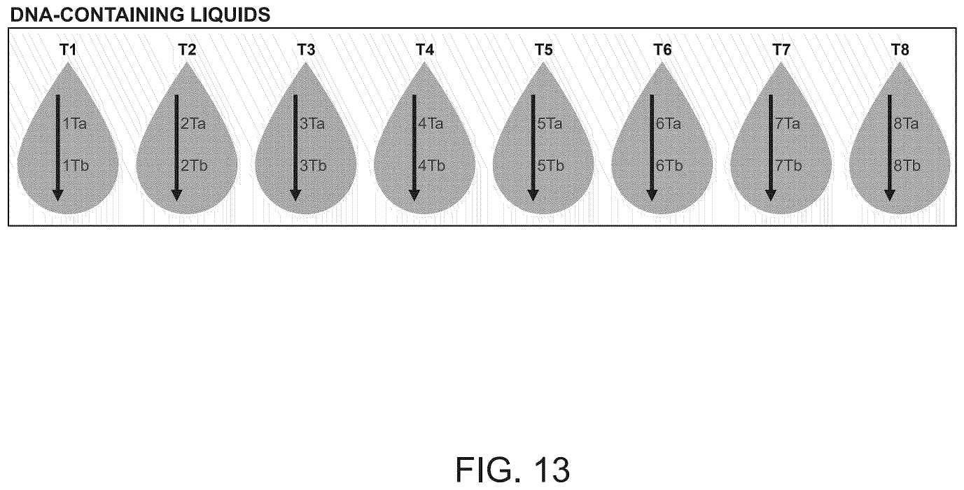

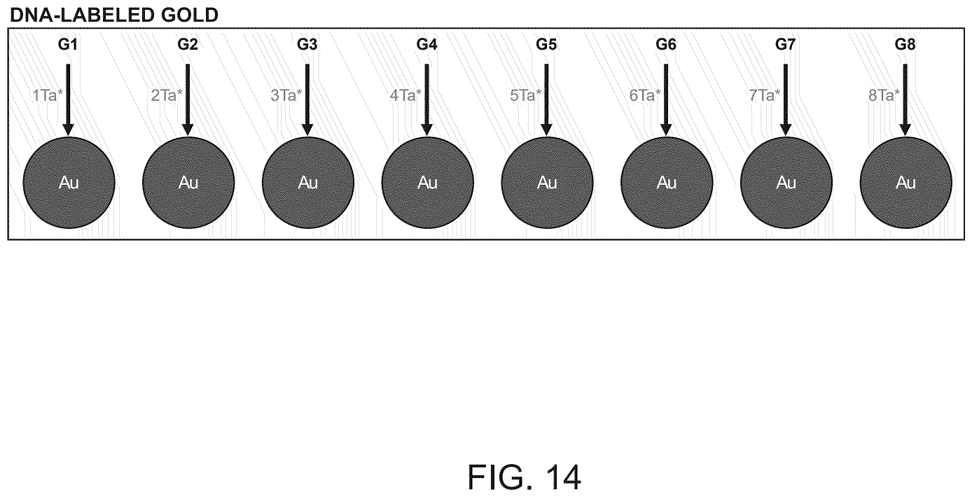

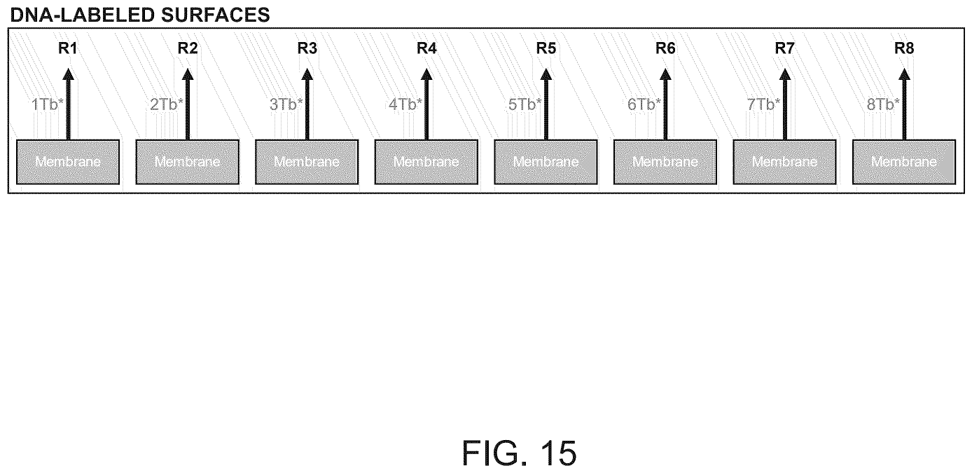

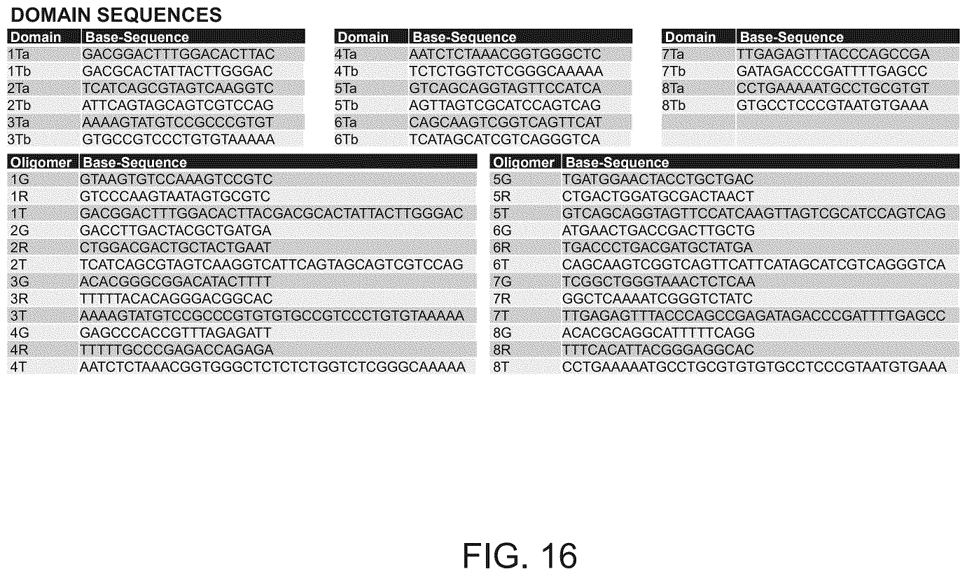

The detection sites 126-140 each include a different compound that is reactive to a different sequence of genetic material. Specifically, in the illustrated embodiment, the detection sites 126-140 include DNA-labeled surfaces as shown in FIG. 15. The detection sites 126-140 may include any compound that detects a particular substance or group of substances as desired. Additionally, the genetic material sample 104 include DNA-containing liquids as shown in FIG. 13 and the gold particle site 124 includes DNA-labeled gold as shown in FIG. 14. FIG. 16 illustrates sample oligomers that may be used for the DNA-labeled surfaces, the DNA-containing liquids, and the DNA-labeled gold.

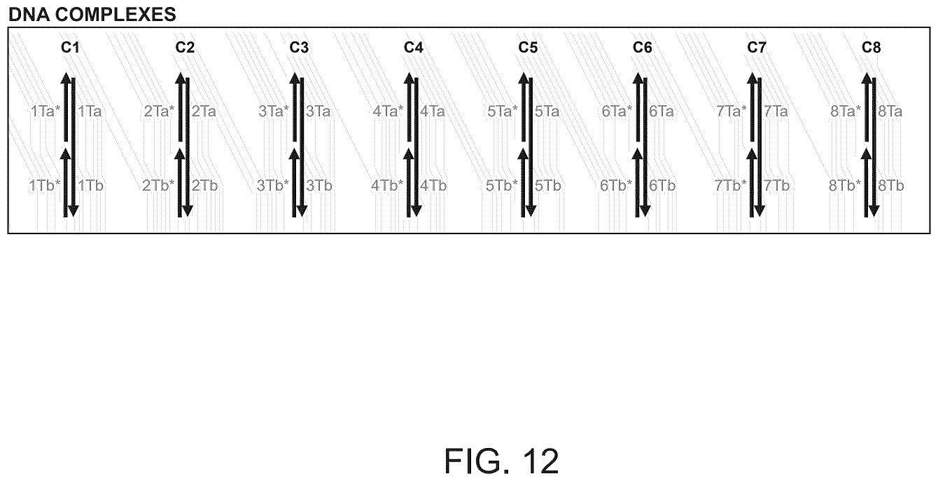

In operation, the sample site 122 is exposed to the genetic material sample 104 to be analyzed by dispensing the genetic material sample 104 on the sample site 122. The genetic material sample 104 flows to the gold particle site 124 where the genetic material attaches to gold nanoparticles within the gold particle site 124. The genetic material then flows to the detection sites 126-140 sequentially and reacts with the substance within the appropriate detection site and changes color. Specifically, the DNA-labeled surfaces, the DNA-containing liquids, and the DNA-labeled gold form a DNA complex as shown in FIG. 12 that changes color. The user then uses the manual 102 to identify which animal the genetic material sample came from based on which detection site 126-140 changed color.

Additionally, the genetic material teaching kit may further include a plurality of items 144 or toys that each correspond to one of the animals to be identified and to one of the genetic material samples 104. The user may then select one of the items 144 once the corresponding genetic material sample 104 has been matched to an animal.

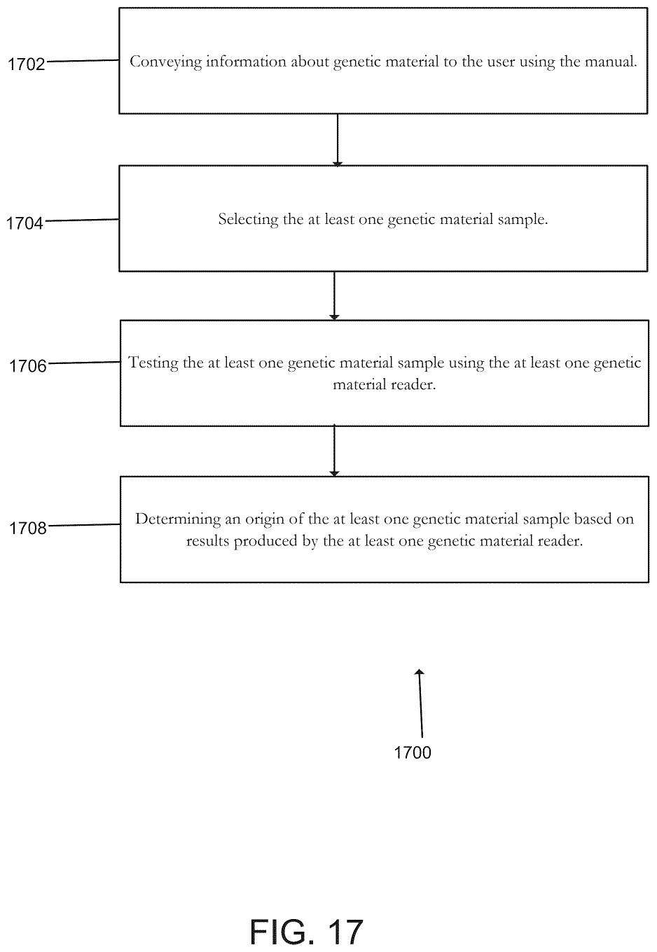

FIG. 17 is a flow diagram of a method 1700 of teaching a user about genetic material using a genetic material teaching kit. The genetic material teaching kit includes a manual, at least one genetic material sample, and at least one genetic material reader. The method 1700 includes conveying 1702 information about genetic material to the user using the manual, selecting 1704 the at least one genetic material sample, testing 1706 the at least one genetic material sample using the at least one genetic material reader, and determining 1708 an origin of the at least one genetic material sample based on results produced by the at least one genetic material reader.

The systems and methods disclosed herein relate to, among other things, a genetic material teaching kit. As described above, teaching the fundamentals of genetic material can be difficult for younger learners. The teaching kit described herein includes a manual, at least one genetic material sample, and at least one genetic material reader and enables younger learners to quickly understand the basics of genetic materials in a fun and easy to understand game or exercise. The teaching kit may further include items, such as small toys, that represent the animals to be identified during the game or teaching exercise. The manual directs the user how to use the teaching kit and explains the basics of genetic materials. The genetic material reader is used to identify the genetic material in each of the genetic material samples such that the user can identify which animal the genetic material sample came from. The user then identifies the animal and may match the corresponding item or toy to the genetic material sample to complete the teaching exercise or game. As such, the genetic material teaching kit describe herein enables the user to understand the fundamentals of genetic material in a fun and simple manner.

Specifically, the genetic material reader described herein may include a lateral flow assay including a plurality of detection sites each including a substance that is configured to react with a specific sequence of genetic material such that the substance can differentiate between different sets of genetic material. In the illustrated embodiment, the substance is configured to change color to indicate that a reaction has taken place. If the substance does not change color, the reaction has not taken place, and the sequence of genetic material is not found in the sample. The genetic material samples are liquid and configured to flow to each of the detection sites sequentially and the genetic material samples may each react with the substance in multiple detection sites. The manual then teaches the user how to interpret the results of the analysis conducted by the lateral flow assay and to identify the animal that each of the genetic material samples corresponds to such that the user understands the fundamentals of genetic material. Other lateral flow assays known in other contexts could be adapted to operate within the principles of the present disclosure and would be suitable for use so long as they could operate as described herein.

In other versions of the kit and method described, the reader does not include a lateral flow assay, but instead includes either other reaction systems using other liquids that simulate lateral flow detection or a pre-arranged pattern to simulate the output of such a system. In some instances, these systems could distinguish samples introduced into the reader based on differences in specific weight of the sample introduced into the reader. In still other versions, introduction of one of the specific samples into the reader would activate a corresponding image to be displayed using an ink system, such as, without limitation, a reactive ink that can change color or appearance when exposed to a corresponding chemical trigger. In some instances, these chemical triggers could be selected from acids, bases, solvents, or other chemical agents known to one of ordinary skill in the art. A user could then correlate the pattern revealed with the pattern indicated in the manual or instructions to identify the origin of the sample.

Accordingly, the genetic material teaching kits described herein enable a user to learn about and understand the basics of genetic material by testing genetic material samples. More specifically, the user reads and learns about genetic material in the manual, uses the understanding gained by reading the manual to test a plurality of different genetic material samples, analyzes the results of the tests, and determines which animal the genetic material corresponds to based on the test conducted as part of the exercise or game. Reading about genetic material and conducting the exercise enables the user to understand the fundamentals of genetic material in a fun and simple manner.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

### Terminology and Interpretative Conventions

Any methods described in the claims or specification should not be interpreted to require the steps to be performed in a specific order unless stated otherwise. Also, the methods should be interpreted to provide support to perform the recited steps in any order unless stated otherwise.

Spatial or directional terms, such as “left,” “right,” “front,” “back,” and the like, relate to the subject matter as it is shown in the drawings. However, it is to be understood that the described subject matter may assume various alternative orientations and, accordingly, such terms are not to be considered as limiting.

Articles such as “the,” “a,” and “an” can connote the singular or plural. Also, the word “or” when used without a preceding “either” (or other similar language indicating that “or” is unequivocally meant to be exclusive—e.g., only one of x or y, etc.) shall be interpreted to be inclusive (e.g., “x or y” means one or both x or y).

The term “and/or” shall also be interpreted to be inclusive (e.g., “x and/or y” means one or both x or y). In situations where “and/or” or “or” are used as a conjunction for a group of three or more items, the group should be interpreted to include one item alone, all the items together, or any combination or number of the items.

The terms have, having, include, and including should be interpreted to be synonymous with the terms comprise and comprising. The use of these terms should also be understood as disclosing and providing support for narrower alternative embodiments where these terms are replaced by “consisting” or “consisting essentially of.”

Unless otherwise indicated, all numbers or expressions, such as those expressing dimensions, physical characteristics, and the like, used in the specification (other than the claims) are understood to be modified in all instances by the term “approximately.” At the very least, and not as an attempt to limit the application of the doctrine of equivalents to the claims, each numerical parameter recited in the specification or claims which is modified by the term “approximately” should be construed in light of the number of recited significant digits and by applying ordinary rounding techniques.

All disclosed ranges are to be understood to encompass and provide support for claims that recite any and all subranges or any and all individual values subsumed by each range. For example, a stated range of 1 to 10 should be considered to include and provide support for claims that recite any and all subranges or individual values that are between and/or inclusive of the minimum value of 1 and the maximum value of 10; that is, all subranges beginning with a minimum value of 1 or more and ending with a maximum value of 10 or less (e.g., 5.5 to 10, 2.34 to 3.56, and so forth) or any values from 1 to 10 (e.g., 3, 5.8, 9.9994, and so forth).

All disclosed numerical values are to be understood as being variable from 0-100% in either direction and thus provide support for claims that recite such values or any and all ranges or subranges that can be formed by such values. For example, a stated numerical value of 8 should be understood to vary from 0 to 16 (100 % in either direction) and provide support for claims that recite the range itself (e.g., 0 to 16), any subrange within the range (e.g., 2 to 12.5) or any individual value within that range (e.g., 15.2).

The terms recited in the claims should be given their ordinary and customary meaning as determined by reference to relevant entries in widely used general dictionaries and/or relevant technical dictionaries, commonly understood meanings by those in the art, etc., with the understanding that the broadest meaning imparted by any one or combination of these sources should be given to the claim terms (e.g., two or more relevant dictionary entries should be combined to provide the broadest meaning of the combination of entries, etc.) subject only to the following exceptions: (a) if a term is used in a manner that is more expansive than its ordinary and customary meaning, the term should be given its ordinary and customary meaning plus the additional expansive meaning, or (b) if a term has been explicitly defined to have a different meaning by reciting the term followed by the phrase “as used in this document shall mean” or similar language (e.g., “this term means,” “this term is defined as,” “for the purposes of this disclosure this term shall mean,” etc.). References to specific examples, use of “i.e.,” use of the word “invention,” etc., are not meant to invoke exception (b) or otherwise restrict the scope of the recited claim terms. Other than situations where exception (b) applies, nothing contained in this document should be considered a disclaimer or disavowal of claim scope.

The subject matter recited in the claims is not coextensive with and should not be interpreted to be coextensive with any embodiment, feature, or combination of features described or illustrated in this document. This is true even if only a single embodiment of the feature or combination of features is illustrated and described in this document.

What is claimed:

1. A genetic material teaching kit comprising:
a manual;
at least one genetic material sample; and
at least one genetic material reader, wherein the at least one genetic material reader performs an analysis on the at least one genetic material sample and a user identifies the at least one genetic material sample based on the analysis and the manual.

2. The genetic material teaching kit of claim 1, wherein the at least one genetic material reader comprises a lateral flow assay.

3. The genetic material teaching kit of claim 2, wherein the lateral flow assay comprises a sample site configured to receive the at least one genetic material sample.

4. The genetic material teaching kit of claim 2, wherein the lateral flow assay comprises a particle site including a plurality of particles.

5. The genetic material teaching kit of claim 4, wherein the plurality of particles are configured to attach to genetic material within the at least one genetic material sample as the at least one genetic material sample flows through the particle site.

6. The genetic material teaching kit of claim 2, wherein the lateral flow assay comprises at least one detection site.

7. The genetic material teaching kit of claim 2, wherein the lateral flow assay comprises a plurality of detection sites.

8. The genetic material teaching kit of claim 7, wherein the plurality of detection sites comprises eight detection sites.

9. The genetic material teaching kit of claim 7, wherein the plurality of detection sites are each configured to change color when each detection site reacts with a specific sequence of genetic material.

10. The genetic material teaching kit of claim 7, wherein the plurality of detection sites are each configured to turn pink or red when each detection site reacts with a specific sequence of genetic material.

11. The genetic material teaching kit of claim 7, wherein the plurality of detection sites each include a substance configured to change color when the substance reacts with a specific sequence of genetic material.

12. The genetic material teaching kit of claim 1, wherein the at least one genetic material reader comprises a base.

13. The genetic material teaching kit of claim 1, wherein the at least one genetic material reader comprises a cover.

14. The genetic material teaching kit of claim 13, wherein the cover comprises an upper cover and a lower cover.

15. The genetic material teaching kit of claim 14, wherein the upper cover comprises at least one opening.

16. The genetic material teaching kit of claim 1, wherein the at least one genetic material reader comprises a multiplex lateral flow assay.

17. The genetic material teaching kit of claim 1, wherein the at least one genetic material sample comprises a deoxyribonucleic acid.

18. The genetic material teaching kit of claim 1, wherein the at least one genetic material sample comprises a synthetic deoxyribonucleic acid.

19. The genetic material teaching kit of claim 1, wherein the at least one genetic material sample comprises a ribonucleic acid.

20. A method of teaching a user about genetic material using a genetic material teaching kit, the genetic material teaching kit comprising a manual, at least one genetic material sample, and at least one genetic material reader, the method comprising:
conveying information about genetic material to the user using the manual;
selecting the at least one genetic material sample;
testing the at least one genetic material sample using the at least one genetic material reader; and
determining an origin of the at least one genetic material sample based on results produced by the at least one genetic material reader.
